(19) United States
(12) Patent Application Publication
(10) Pub. No.: US 2023/0245781 A1
(43) Pub. Date: Aug. 3, 2023

(54) System and Method for Personal Health Analytics Technical Field

(71) Applicant: Ovuline, Inc., Boston, MA (US)

(72) Inventors: Alex Baron, Canton, MA (US); Gina Nebesar, Boston, MA (US); Vasile Tofan, Chisinau, MD (US); Paris Wallace, Cambridge, MA (US)

(73) Assignee: Ovuline, Inc., Boston, MA (US)

(21) Appl. No.: 18/296,700

(22) Filed: Apr. 6, 2023

Related U.S. Application Data
(63) Continuation of application No. 14/517,046, filed on Oct. 17, 2014, now abandoned.
(60) Provisional application No. 61/891,980, filed on Oct. 17, 2013.

(51) Int. Cl. G16H 50/30; G16Z 99/00

(52) U.S. Cl. CPC G16H 50/30; G16Z 99/00; G16H 10/60

(57) ABSTRACT

Disclosed is a system and method for providing personalized health information. The disclosed systems and methods provide for analyzing self-reported data relating to personal health information in order to predict a health condition. The systems and methods disclosed provide personalized interpretations of medical knowledge in light of the growing collection of personal health information that is publicly and privately available. Accordingly, the present disclosure provides systems and methods for personalized information intermediation to help individuals to navigate the growing selection of personal health products and services, and to contribute to health care system efficiencies by improving individual health knowledge. In some embodiments, the systems and methods disclosed provide fertility prediction encompassing a predicted date of ovulation and fertility window.

### RELATED APPLICATIONS

This application is a continuation of U.S. Pat. Application No. 14/517,046, filed Oct. 17, 2014, and claims priority to and the benefit of U.S. Provisional Pat. Application No. 61/891,980, filed Oct. 17, 2013, the entirety of both of which are incorporated by reference herein.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever

### FIELD

The present disclosure relates generally towards predicting a health event, and more particularly, to analytical information systems and methods for providing personalized health information.

### RELATED ART

Clinical guidelines are typically interpreted by medical professionals to provide treatment instructions in response to a wide range of diagnosable or foreseeable health events. These health events can be states of aging, pathology, or mere health risks. The interpretation of clinical guidelines is performed in accordance with professional training and reflects the individual experience of those medical professionals. As such, the ability of medical professionals to correctly interpret clinical guidelines for a particular individual can be limited by the number of cases and the peculiar health event manifestations that have been studied or observed. In other words, medical professionals arc limited by the human cognitive capacity for pattern recognition, memory, and reasoning and their interpretation of clinical guidelines can be improved.

### SUMMARY

With the advent of personal health and wellness data gathering (e.g. FitBit® devices, 23andMe™ personal genetic testing), there is a need for providing personalized interpretations of medical knowledge in light of this growing collection of personal health information. Similarly, there is a need for providing personalized information intermediation to help individuals to navigate the growing selection of personal health products and services, and to contribute to health care system efficiencies by improving individual health knowledge.

In accordance with one or more embodiments, the present disclosure discloses a method for providing personalized health information. As discussed herein, the embodiments of the disclosed method involve analyzing self-reported data relating to personal health information to predict a health condition.

In accordance with one or more embodiments, a non-transitory computer-readable storage medium is provided, the computer-readable storage medium tangibly storing thereon, or having tangibly encoded thereon, computer readable instructions that when executed cause at least one processor to perform a method for providing personalized health information.

In accordance with one or more embodiments, a system is provided that comprises one or more computing devices configured to provide functionality in accordance with such embodiments. In accordance with one or more embodiments, functionality is embodied in steps of a method performed by at least one computing device. In accordance with one or more embodiments, program code to implement functionality in accordance with one or more such embodiments is embodied in, by and/or on a computer-readable medium.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a schematic diagram illustrating an example of a network within which the systems and methods disclosed herein could be implemented according to some embodiments of the present disclosure;

FIG. 2 depicts a schematic diagram illustrating a client device in accordance with some embodiments of the present disclosure;

FIG. 3 depicts a system for providing personalized health prediction in accordance with some embodiments of the present disclosure;

FIG. 4 depicts a method for providing personalized health prediction in accordance with some embodiments of the present disclosure;

FIG. 5A depicts a method for providing a personalized interpretation of clinical guidelines in accordance with some embodiments of the present disclosure;

FIG. 5B depicts a method for providing a personalized interpretation of clinical guides in accordance with some embodiments of the present disclosure;

FIG. 6 depicts a method for providing a personalized information filtering in accordance with some embodiments of the present disclosure;

FIG. 7 depicts an exemplary fertility predictive application in accordance with some embodiments of the present disclosure; and

FIG. 8 is a block diagram illustrating architecture of a hardware device in accordance with one or more embodiments of the present disclosure.

### DETAILED DESCRIPTION

The present disclosure will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not neccessarily refer to the same emodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer, special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

These computer program instructions can be provided to a processor of a general purpose computer, special purpose computer, ASIC, or other programmable data processing apparatus, such that the instructions, which execute via the processor of the computer or other programmable data processing apparatus, implement the functions/acts specified in the block diagrams or operational block or blocks.

For the purposes of this disclosure a computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Servers may vary widely in configuration or capabilities, but generally a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network. Various types of devices may, for example, be made available to provide an interoperable capability for differing architectures or protocols. As one illustrative example, a router may provide a link between otherwise separate and independent LANs.

A communication link or channel may include, for example, analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art. Furthermore, a computing device or other related electronic devices may be remotely coupled to a network, such as via a telephone line or link, for example.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further include a system of terminals, gateways, routers, or the like coupled by wireless radio links, or the like, which may move freely, randomly or organize themselves arbitrarily, such that network topology may change, at times even rapidly. A wireless network may further employ a plurality of network access technologies, including Long Term Evolution (LTE), WLAN, Wireless Router (WR) mesh, or 2nd, 3rd, or 4th generation (2G, 3G, or 4G) cellular technology, or the like. Network access technologies may enable wide area coverage for devices, such as client devices with varying degrees of mobility, for example.

For example, a network may enable RF or wireless type communication via one or more network access technologies, such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, or the like. A wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like. Servers may vary widely in configuration or capabilities, but generally a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For purposes of this disclosure, a client (or consumer or user) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device an Near Field Communication (NFC) device, a Personal Digital Assistant (PDA), a handheld computer, a tablet computer, a laptop computer, a set top box, a wearable computer, an integrated device combining various features, such as features of the forgoing devices, of the like.

A client device may vary in terms of capabilities or features. Claimed subject matter is intended to cover a wide range of potential variations. For example, a cell phone may include a numeric keypad or a display of limited functionality, such as a monochrome liquid crystal display (LCD) for displaying text. In contrast, however, as another example, a web-enabled client device may include one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

A client device may include or may execute a variety of operating systems, including a personal computer operating system, such as a Windows, iOS or Linux, or a mobile operating system, such as iOS, Android, or Windows Mobile, or the like. A client device may include or may execute a variety of possible applications, such as a client software application enabling communication with other devices, such as communicating one or more messages, such as via email, short message service (SMS), or multimedia message service (MMS), including via a network, such as a social network, including, for example, Facebook®, LinkedIn®, Twitter®, Flickr®, or Google+®, Instagram™, to provide only a few possible examples. A client device may also include or execute an application to communicate content, such as, for example, textual content, multimedia content, or the like. A client device may also include or execute an application to perform a variety of possible tasks, such as browsing, searching, playing various forms of content, including locally stored or streamed video, or games. The foregoing is provided to illustrate that claimed subject matter is intended to include a wide range of possible features or capabilities.

The principles described herein may be embodied in many different forms. As discussed herein, the present disclosure provides systems and methods for providing personalized health information. The disclosed systems and methods provide embodiments for analyzing self-reported data relating to personal health information in order to predict a health condition. According to some embodiments, the data relating to personal health information may be automatically retrieved, received and/or downloaded from a data store housing such information. The systems and methods disclosed provide embodiments for personalized interpretations of medical knowledge in light of the growing collection of personal health information that is publicly and privately available. Accordingly, the present disclosure discloses embodiments for systems and methods for personalized information intermediation to help individuals to navigate the growing selection of personal health products and services, and to contribute to health care system efficiencies by improving individual health knowledge. In some embodiments, as discussed in more detail below, the systems and methods provide fertility prediction encompassing a predicted date of ovulation and fertility window.

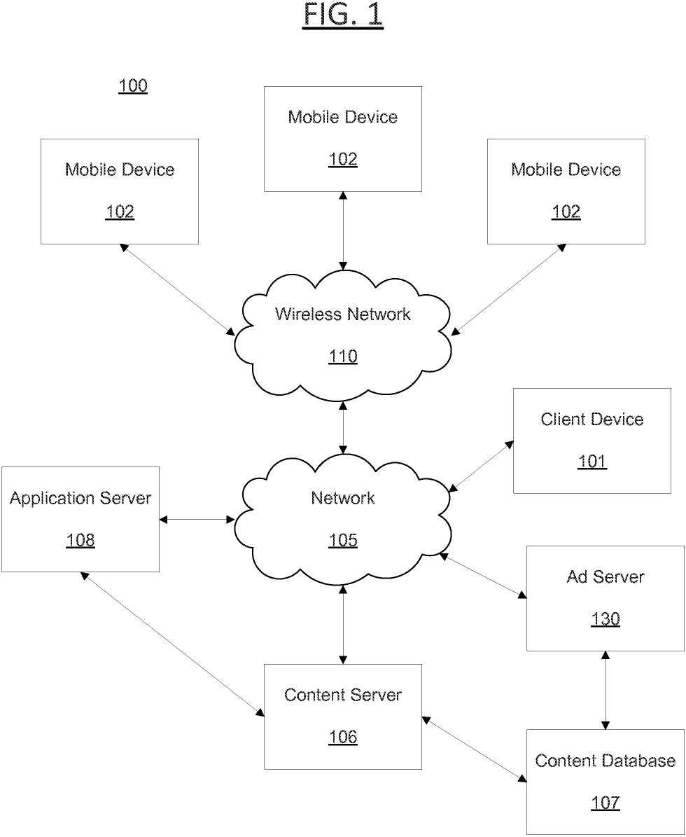

Certain embodiments will now be described in greater detail with reference to the figures. In general, with reference to FIG. 1, a system 100 in accordance with an embodiment of the present disclosure is shown. FIG. 1 shows components of a general environment in which the systems and methods discussed herein may be practiced. Not all the components may be required to practice the disclosure, and variations in the arrangement and type of the components may be made without departing from the spirit or scope of the disclosure. As shown, system 100 of FIG. 1 includes local area networks (“LANs”)/wide area networks (“WANs”) – network 105, wireless network 110, mobile devices (client devices) 102-104 and client device 101. FIG. 1 additionally includes a variety of servers, such as content server 106, application (or “App”) server 108, and advertising (“ad”) server 130.

One embodiment of mobile devices 102-103 is described in more detail below. Generally, however, mobile devices 102-104 may include virtually any portable computing device capable of receiving and sending a message over a network, such as network 105, wireless network 110, or the like. Mobile devices 102-104 may also be described generally as client devices that are configured to be portable. Thus, mobile devices 102-104 may include virtually any portable computing device capable of connecting to another computing device and receiving information. Such devices include multi-touch and portable devices such as, cellular telephones, smart phones, display pagers, radio frequency (RF) devices, infrared (IR) devices, Personal Digital Assistants (PDAs), handheld computers, laptop computers, wearable computers, tablet computers, integrated devices combining one or more of the preceding devices, and the like. As such, mobile devices 102-104 typically range widely in terms of capabilities and features. For example, a cell phone may have a numeric keypad and a few lines of monochrome LCD display on which only text may be displayed. In another example, a web-enabled mobile device may have a touch sensitive screen, a stylus, and several lines of color LCD display in which both text and graphics may be displayed.

A web-enabled mobile device may include a browser application that is configured to receive and to send web pages, web-based messages, and the like. The browser application may be configured to receive and display graphics, text, multimedia, and the like, employing virtually any web based language, including a wireless application protocol messages (WAP), and the like. In one embodiment, the browser application is enabled to employ Handheld Device Markup Language (HDML), Wireless Markup Language (WML), WMLScript, JavaScript, Standard Generalized Markup Language (SMGL), HyperText Markup Language (HTML), extensible Markup Language (XML), and the like, to display and send a message.

Mobile devices 102-104 also may include at least one client application that is configured to receive content from another computing device. The client application may include a capability to provide and receive textual content, graphical content, audio content, and the like. The client application may further provide information that identifies itself, including a type, capability, name, and the like. In one embodiment, mobile devices 102-104 may uniquely identify themselves through any of a variety of mechanisms, including a phone number, Mobile Identification Number (MIN), an electronic serial number (ESN), or other mobile device identifier.

In some embodiments, mobile devices 102-104 may also communicate with non-mobile client devices, such as client device 101, or the like. In one embodiment, such communications may include sending and/or receiving messages, share photographs, audio clips, video clips, or any of a variety of other forms of communications. Client device 101 may include virtually any computing device capable of communicating over a network to send and receive information. The set of such devices may include devices that typically connect using a wired or wireless communications medium such as personal computers, multiprocessor systems, microprocessor-based or programmable consumer electronics, network PCs, or the like. Thus, client device 101 may also have differing capabilities for displaying navigable views of information.

Client devices 101-104 computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

Wireless network 110 is configured to couple mobile devices 102-104 and its components with network 105. Wireless network 110 may include any of a variety of wireless sub-networks that may further overlay stand-alone ad-hoc networks, and the like, to provide an infrastructure-oriented connection for mobile devices 102-104. Such sub-networks may include mesh networks, Wireless LAN (WLAN) networks, cellular networks, and the like.

Wireless network 110 may further include an autonomous system of terminals, gateways, routers, and the like connected by wireless radio links, and the like. These connectors may be configured to move freely and randomly and organize themselves arbitrarily, such that the topology of wireless network 110 may change rapidly. Wireless network 110 may further employ a plurality of access technologies including 2nd (2G), 3rd (3G), and/or 4th (4G) generation radio access for cellular systems, WLAN, Wireless Router (WR) mesh, and the like. Access technologies such as 2G, 3G, 4G and future access networks may enable wide area coverage for mobile devices, such as mobile devices 102-104 with various degrees of mobility. For example, wireless network 110 may enable a radio connection through a radio network access such as Global System for Mobil communication (GSM), General Packet Radio Services (GPRS), Enhanced Data GSM Environment (EDGE), Wideband Code Division Multiple Access (WCDMA), and the like. In essence, wireless network 110 may include virtually any wireless communication mechanism by which information may travel between mobile device s 102-104 and another computing device, network, and the like.

Network 105 is configured to couple content server 106, application server 108, or the like, with other computing devices, including, client device 101, and through wireless network 110 to mobile devices 102-104. Network 105 is enabled to employ any form of computer readable media for communicating information from one electronic device to another. Also, network 105 can include the Internet in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router acts as a link between LANs, enabling messages to be sent from one to another. Also, communication links within LANs typically include twisted wire pair or coaxial cable, while communication links between networks may utilize analog telephone lines, full or fractional dedicated digital lines including T1, T2, T3, and T4, Integrated Services Digital Networks (ISDNs), Digital Subscriber Lines (DSLs), wireless links including satellite links, or other communications links known to those skilled in the art. Furthermore, remote computers and other related electronic devices could be remotely connected to either LANs or WANs via a modem and temporary telephone link. In essence, network 105 includes any communication method by which information may travel between content server 106, application server 108, client device 101, and/or other computing devices.

Within the communications networks utilized or understood to be applicable to the present disclosure, such networks will employ various protocols that are used for communication over the network. Signal packets communicated via a network, such as a network of participating digital communication networks, may be compatible with or compliant with one or more protocols. Signaling formats or protocols employed may include, for example, TCP/IP, UDP, DECnet, NetBEUI, IPX, APPLETALK™, or the like. Versions of the Internet Protocol (IP) may include IPv4 or IPv6. The Internet refers to a decentralized global network of networks. The Internet includes local area networks (LANs), wide area networks (WANs), wireless networks, or long haul public networks that, for example, allow signal packets to be communicated between LANs. Signal packets may be communicated between nodes of a network, such as, for example, to one or more sites employing a local network address. A signal packet may, for example, be communicated over the Internet from a user site via an access node coupled to the Internet. Likewise, a signal packet may be forwarded via network nodes to a target site coupled to the network via a network access node, for example. A signal packet communicated via the Internet may, for example, be routed via a path of gateways, servers, etc. that may route the signal packet in accordance with a target address and availability of a network path to the target address.

According to some embodiments, the present disclosure may also be utilized within or in conjunction with a social network and/or a social networking site. A social network refers generally to a network of individuals, such as acquaintances, friends, family, colleagues, or co-workers, coupled via a communications network or via a variety of sub-networks. Potentially, additional relationships may subsequently be formed as a result of social interaction via the communications network or sub-networks. In some embodiments, multi-modal communications may occur between members of the social network. Individuals within one or more social networks may interact or communication with other members of a social network via a variety of devices. Multi-modal communication technologies refers to a set of technologies that permit interoperable communication across multiple devices or platforms, such as cell phones, smart phones, tablet computing devices, personal computers, televisions, set-top boxes, SMS/MMS, email, instant messenger clients, forums, social networking sites, or the like.

In some embodiments, the disclosed networks 110 and/or 105 may comprise a content distribution network(s). A “content delivery network” or “content distribution network” (CDN) generally refers to a distributed content delivery system that comprises a collection of computers or computing devices linked by a network or networks. A CDN may employ software, systems, protocols or techniques to facilitate various services, such as storage, caching, communication of content, or streaming media or applications. A CDN may also enable an entity to operate or manage another’s site infrastructure, in whole or in part.

The content server 106 may include a device that includes a configuration to provide content via a network to another device. A content server 106 may, for example, host a site or service, such as an email platform, social networking site music site/platform, a movie site or platform or any other type of content hosted, retrievable, downloadable or accessible via a web page or service, or a personal user site (such as a blog, vlog, online dating site, and the like). Indeed, a content server 106 may also host a variety of sites providing any range of content, including, but not limited to, music sites, movie sites, streaming content, business sites, educational sites, dictionary sites, encyclopedia sites, wikis, financial sites, government sites, and the like. In some embodiments, the content server 106 may also provide advertising or marketing content. Devices that may operate as content server 106 include personal computers desktop computers, multiprocessor systems, microprocessor-based or programmable consumer electronics, network PCs, servers, and the like.

Content server 106 can further provide a variety of services that include, but are not limited to, email services, photo services, web services, third-party services, audio services, video services, email services, instant messaging (IM) services, SMS services, MMS services, FTP services, voice over IP (VOIP) services, or the like. Such services, for example the email services and email platform, can be provided via the content server 106. Examples of content may include images, text, audio, video, or the like, which may be processed in the form of physical signals, such as electrical signals, for example, or may be stored in memory, as physical states, for example.

An ad server 130 comprises a server that stores online advertisements for presentation to users. “Ad serving” refers to methods used to place online advertisements on websites, in applications, or other places where users are more likely to see them, such as during an online session or during computing platform use, for example, Various monetization techniques or models may be used in connection with sponsored advertising, including advertising associated with user. Such sponsored advertising includes monetization techniques including sponsored search advertising, non-sponsored search advertising, guaranteed and non-guaranteed delivery advertising, ad networks/exchanges, ad targeting, ad serving and ad analytics.

For example, a process of buying or selling online advertisements may involve a number of different entities, including advertisers, publishers, agencies, networks, or developers. To simplify this process, organization systems called “ad exchanges” may associate advertisers or publishers, such as via a platform to facilitate buying or selling of online advertisement inventory from multiple ad networks. “Ad networks” refers to aggregation of ad space supply from publishers, such as for provision en masse to advertisers. For web portals, advertisements may be displayed on web pages resulting from a user-defined search based at least in part upon one or more search terms. Advertising may be beneficial to users, advertisers or web portals if displayed advertisements are relevant to interests of one or more users. Thus, a variety of techniques have been developed to infer user interest, user intent or to subsequently target relevant advertising to users. One approach to presenting targeted advertisements includes employing demographic characteristics (e.g., age, income, sex, occupation, etc.) for predicting user behavior, such as by group. Advertisements may be presented to users in a targeted audience based at least in part upon predicted user behavior(s). Another approach includes profile-type ad targeting. In this approach, user profiles specific to a user may be generated to model user behavior, for example, by tracking a user’s path through a web site or network of sites, and compiling a profile based at least in part on pages or advertisements ultimately delivered. A correlation may be identified, such as for user purchases, for example. An identified correlation may be used to target potential purchasers by targeting content or advertisements to particular users. During presentation of advertisements, a presentation system may collect descriptive content about types of advertisements presented to users. A broad range of descriptive content may be gathered, including content specific to an advertising presentation system. Advertising analytics gathered may be transmitted to locations remote to an advertising presentation system for storage or for further evaluation. Where advertising analytics transmittal is not immediately available, gathered advertising analytics may be stored by an advertising presentation system until transmittal of those advertising analytics becomes available.

Servers 106, 108 and 130 may be capable of sending or receiving signals, such as via a wired or wireless network, or may be capable of processing or storing signals, such as in memory as physical memory states. Devices capable of operating as a server may include, as examples, dedicated rack-mounted servers, desktop computers, laptop computers, set top boxes, integrated devices combining various features, such as two or more features of the foregoing devices, or the like. Servers may vary widely in configuration or capabilities, but generally, a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

In an embodiment, users are able to access services provided by servers 106, 108 and/or 130. This may include in a non-limiting example, email servers, social networking services servers, SMS servers, IM servers, MMS servers, exchange servers, photo-sharing services servers, and travel services servers, via the network 105 using their various devices 101-104. In some embodiments, applications, such as, but not limited to the personal fertility analytics application discussed herein, can be hosted by the application server 108. Thus, the application server 108 can store various types of applications and application related information including application data and user profile information. In another example, a content server 106 acting as an email server can host email applications; therefore, the content server 106 can store various types of applications and application related information including email application data and user profile information, which can be correlated with the application server 108. It should also be understood that content server 106 can also store various types of data related to the content and services provided by content server 106 in an associated content database 107, as discussed in more detail below. Embodiments exist where the network 105 is also coupled with/connected to a Trusted Search Server (TSS) which can be utilized to render content in accordance with the embodiments discussed herein.

Moreover, although FIG. 1 illustrates servers 106, 108 and 130 as single computing devices, respectively, the disclosure is not so limited. For example, one or more functions of servers 106, 108 and/or 130 may be distributed across one or more distinct computing devices. Moreover, in one embodiment, servers 106, 108 and/or 130 may be integrated into a single computing device, without departing from the scope of the present disclosure.

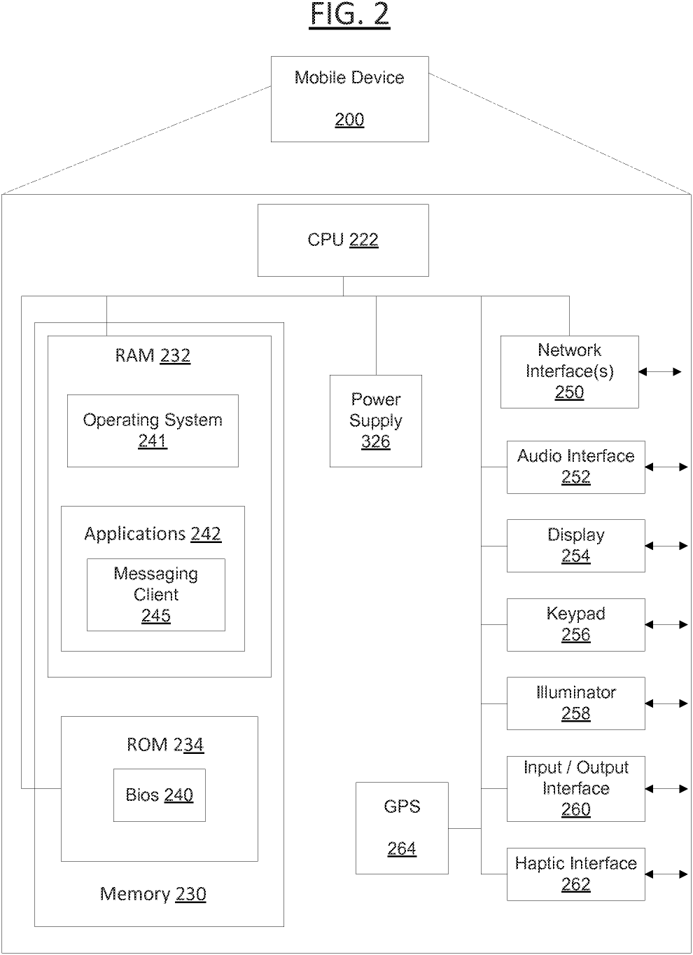

FIG. 2 is a schematic diagram illustrating a client device showing an example embodiment of a client device that may be used within the present disclosure. Client device 200 may include many more or less components than those shown in FIG. 2. However, the components shown are sufficient to disclose an illustrative embodiment for implementing the present disclosure. Client device 200 may represent, for example, client devices discussed above in relation to FIG. 1.

As shown in the figure, Client device 200 includes a processing unit (CPU) 222 in communication with a mass memory 230 via a bus 224. Client device 200 also includes a power supply 226, one or more network interfaces 250, an audio interface 252, a display 254, a keypad 256, an illuminator 258, an input/output interface 260, a haptic interface 262, and an optional global positioning systems (GPS) receiver 264. Power supply 226 provides power to Client device 200. A rechargeable or non-rechargeable battery may be used to provide power. The power may also be provided by an external power source, such as an AC adapter or a powered docking cradle that supplements and/or recharges a battery.

Client device 200 may optionally communicate with a base station (not shown), or directly with another computing device. Network interface 250 includes circuitry for coupling Client device 200 to one or more networks, and is constructed for use with one or more communication protocols and technologies including, but not limited to, global system for Client communication (GSM), code division multiple access (CDMA), time division multiple access (TDMA), user datagram protocol (UDP), transmission control protocol/Internet protocol (TCP/IP), SMS, general packet radio service (GPRS), WAP, ultra wide band (UWB), IEEE 802.16 Worldwide Interoperability for Microwave Access (WiMax), SIP/RTP, or any of a variety of other wireless communication protocols. Network interface 250 is sometimes known as a transceiver, transceiving device, or network interface card (NIC).

Audio interface 252 is arranged to produce and receive audio signals such as the sound of a human voice. For example, audio interface 252 may be coupled to a speaker and microphone (not shown) to enable telecommunication with others and/or generate an audio acknowledgement for some action. Display 254 may be a liquid crystal display (LCD), gas plasma, light emitting diode (LED), or any other type of display used with a computing device. Display 254 may also include a touch sensitive screen arranged to receive input from an object such as a stylus or a digit from a human hand.

Keypad 256 may comprise any input device arranged to receive input from a user. For example, keypad 256 may include a push button numeric dial, or a keyboard. Keypad 256 may also include command buttons that are associated with selecting and sending images. Illuminator 258 may provide a status indication and/or provide light. Illuminator 258 may remain active for specific periods of time or in response to events. For example, when illuminator 258 is active, it may backlight the buttons on keypad 256 and stay on while the client device is powered. Also, illuminator 258 may backlight these buttons in various patterns when particular actions are performed, such as dialing another client device. Illuminator 258 may also cause light sources positioned within a transparent or translucent case of the client device to illuminate in response to actions.

Client device 200 also comprises input/output interface 260 for communicating with external devices, such as a headset, or other input or output devices not shown in FIG. 2. Input/output interface 260 can utilize one or more communication technologies, such as USB, infrared, Bluetooth™, or the like. Haptic interface 262 is arranged to provide tactile feedback to a user of the client device. For example, the haptic interface may be employed to vibrate client device 200 in a particular way when the Client device 200 receives a communication from another user.

Optional GPS transceiver 264 can determine the physical coordinates of Client device 200 on the surface of the Earth, which typically outputs a location as latitude and longitude values. GPS transceiver 264 can also employ other geo-positioning mechanisms, including, but not limited to, triangulation, assisted GPS (AGPS), E-OTD, CI, SAI, ETA, BSS or the like, to further determine the physical location of Client device 200 on the surface of the Earth. It is understood that under different conditions, GPS transceiver 264 can determine a physical location within millimeters for Client device 200; and in other cases, the determined physical location may be less precise, such as within a meter or significantly greater distances. In one embodiment, however, Client device may through other components, provide other information that may be employed to determine a physical location of the device, including for example, a MAC address, IP address, or the like.

Mass memory 230 includes a RAM 232, a ROM 234, and other storage means. Mass memory 230 illustrates another example of computer storage media for storage of information such as computer readable instructions, data structures, program modules or other data. Mass memory 230 stores a basic input/output system (“BIOS”) 240 for controlling low-level operation of Client device 200. The mass memory also stores an operating system 241 for controlling the operation of Client device 200. It will be appreciated that this component may include a general purpose operating system such as a version of UNIX, or LINUX™, or a specialized client communication operating system such as Windows Client™, or the Symbian® operating system. The operating system may include, or interface with a Java virtual machine module that enables control of hardware components and/or operating system operations via Java application programs.

Memory 230 further includes one or more data stores, which can be utilized by Client device 200 to store, among other things, applications 242 and/or other data. For example, data stores may be employed to store information that describes various capabilities of Client device 200. The information may then be provided to another device based on any of a variety of events, including being sent as part of a header during a communication, sent upon request, or the like. At least a portion of the capability” information may also be stored on a disk drive or other storage medium (not shown) within Client device 200.

Applications 242 may include computer executable instructions which, when executed by Client device 200, transmit, receive, and/or otherwise process audio, video, images, and enable telecommunication with another user of another client device. Other examples of application programs include calendars, browsers, contact managers, task managers, transcoders, database programs, word processing programs, security applications, spreadsheet programs, games, search programs, and so forth. Applications 242 may further include messaging client 245 that is configured to send, to receive, and/or to otherwise process messages using SMS, MMS, IM, email, VOIP, and/or any of a variety of other messaging communication protocols. Although a single messaging client 245 is illustrated it should be clear that multiple messaging clients may be employed. For example, one messaging client may be configured to manage SMS messages, where another messaging client manages IM messages, and yet another messaging client is configured to manage serving advertisements, emails, or the like.

Having described the components of the general architecture employed within the disclosed systems and methods, the components’ general operation with respect to the disclosed systems and methods will now be described.

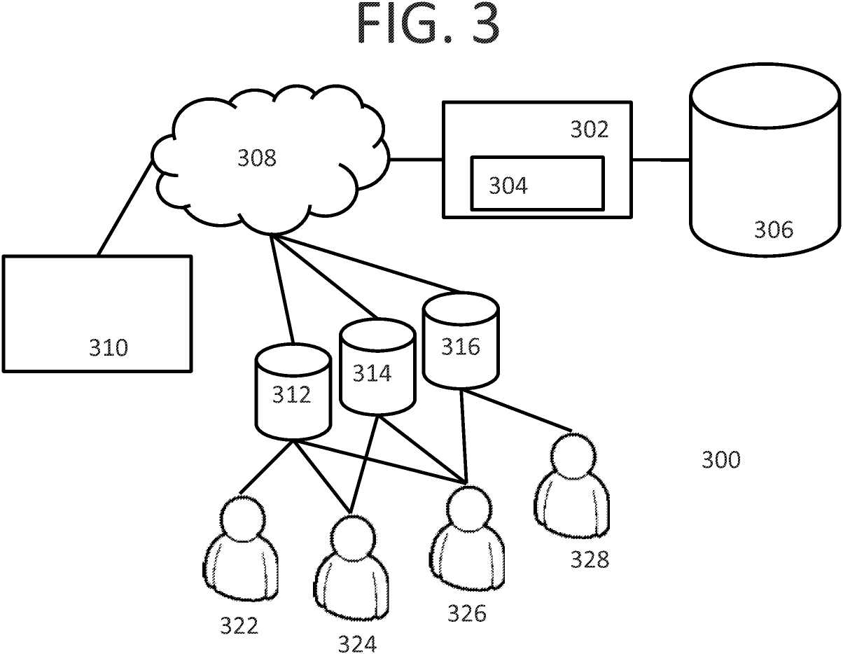

FIG. 3 depicts a block diagram of a personal health analytics system. 300 according to an embodiment. The components of FIG. 1 can be implemented in conjunction with at least a portion, if not all of the components of the general architecture discussed above in relation to FIGS. 1 and 2. The system 300 can include computer system 302, local database 306, network 308, remote information system 310, and personal health information databases 312, 314, and 316. The computer system 302 also includes analytics application 304. The computer system 302 also includes the personal health information databases 312, 314 and 316 being directly connected to devices 322-328, as illustrated in FIG. 3. Indeed, devices 322-328 may be related to the client devices, users and/or servers discussed above. The components of system 300, such as but not limited to computer system 302, local database 306, remote information system 310, and personal health information databases 312, 314 and 316, as well as analytics application 304 could be hosted by a web server, content provider, application service provider, advertisements server, a user’s computing device, and/or any combination thereof. Indeed, it should be understood by those of skill in the art that the components of system 300 discussed herein are non-exhaustive, as additional or fewer components and allocations to device structures (e.g., servers or computing devices) may be applicable to the embodiments of the systems and methods discussed herein.

As discussed in more detail below, the personal health analytics application 104, in various embodiments, can combine predictive statistical modeling of personal health information with clinical knowledge representation techniques to determine future risks of a particular health event within a predetermined population and/or set of patients.

### Predictive Analytics for Personal Health

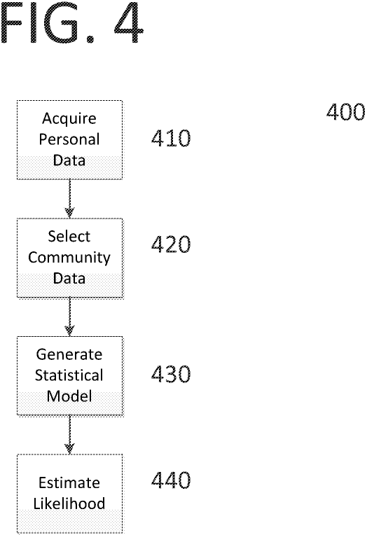

According one embodiment of the present disclosure, as shown in FIG. 4, a process 400 is provided for analyzing self-reported data relating to personal health information to predict a health condition. First, in step 410, a plurality of personal datasets can be acquired through the use of personal electronic devices. For example, electronic fitness tracking devices such as, but not limited to, a FitBit® device (among other know or to be known devices of similar scope) can be directed by the user to automatically transmit tracked fitness or activity data to be aggregated into a personal dataset, via a smartphone or a web interface. According to some embodiments, personal datasets can be automatically retrieved using known or to be known techniques for acquiring data from users and/or data sources housing such information.

According to one embodiment, step 410 can further include periodically posing to the voluntary participants, through a user interface on a personal electronic device, a first set of questions designed to obtain a time series of variable health attributes. The step 410 can further include posing a second set of questions designed to obtain a status of a past or current health event. For example, a variable health attribute can be a daily activity measure through a FitBit® device, or an otherwise self-reported mood state from the participants. Since these health attributes can vary over time, a consistent user interface can be provided to the participants. As a way of engaging and motivating the participants, a visualization of the participant submitted answers to the periodically posed questions can be provided to an information feedback. Similarly, for each of the questions in the second set of questions designed to obtain a status of a past or current health event from the participants, a personalized informational feedback can be provided to the participant to encourage a further response(s).

According to one embodiment, the self-reported personal health attribute used to provide a personal fertility analytics application can be one of a mood quality, a menstrual cycle type, a menstrual period length, date of last period, menstrual period dates, spotting dates, cervical fluid quality, intercourse dates, ovulation test results, pregnancy test results, body basal temperature, number of steps walked, health, quality, weight, medications taken, nutrition consumed, time slept, blood pressure, physical activity, other relevant notes, and/or any combination thereof.

According to another embodiment, the self-reported personal health attribute used to provide a personal pregnancy analytics application can be one of number of steps walked, mood quality, health quality, weight, medications taken, nutrition consumed, time slept, blood pressure, activity, baby kick counts, contraction frequency, tagged comments, attached relevant photos, other relevant notes, and/or any combination thereof.

Next, in step 420, a community dataset relating to a health event can be selected from the plurality of personal datasets. For example, a subset of all participants can be grouped into a virtual community according to their responses to the questions posed in step 410. As such, the community dataset can contain personal health information from a community of participants having in common a similar past or current health event. Furthermore, a plurality of such virtual communities can be created by groupings according to one or more health events. According to one embodiment, in a fertility prediction application, an entire set of participants can be grouped, or filtered, according to a combination of menstruation regularity and participant age. The filtered groups can be further grouped, or filtered, according to menstrual cycle length, menstrual duration and the like. According to another embodiment, the filtered groups can be further grouped, or filtered, according to a geographical location.

Next, in step 430, a statistical model of the health event can be generated from the selected community dataset. According to one embodiment, a statistical model can be generated for a grouped, or filtered, set of participants having a similar age and/or regular menstruation cycles. According to another embodiment, a statistical model can be generated for the participants having a similar age and/or irregular menstruation cycles. It can be appreciated that by generating a distinct statistical model for distinctive groups of participants, a number of predictive health attributes can be determined for each of the groups of participants. For example, for a group of participants having irregular menstruations, a menstruation duration can be eliminated as a predictive health attribute. According to other embodiments, a predictive health attribute can be discovered or added to the generated model.

According to one embodiment, the step 430 for generating a statistical model can include a step of extracting a sequence health attributes for an individual participant. Optionally, a personalized statistical model can be generated from each individual participant from the extracted sequence health attributes.

Finally, in step 440, a likelihood of a personal health attribute relating to the health event can be estimated using the generated statistical model. According to one embodiment, the step 440 can include a step of determining a threshold in a model parameter of the generated personal statistical model. Here, the threshold separates a sequence of health attributes likely to be associated with the personal health attribute from a sequence of health attributes unlikely to be associated with the personal health attribute. As such, the model parameter for the individual participant according to the extracted sequence can be estimated, and the individual participant can be classified according to the determined threshold.

According to one embodiment, in a fertility predictive application, a date of ovulation and a fertility window can be predicted. First, a likelihood of ovulation can be estimated for five consecutive days. For example, a likelihood of ovulation can be estimated for the day of prediction and four following days. A plurality of health attributes can be correlated with a date of ovulation, and a probability, or likelihood, of ovulation for each of the five days can be determined based from the correlations of the plurality of health attributes and the ovulation date. Next, a maximum probability, or likelihood, for ovulation is determined among the five consecutive days, and the day of maximum probability or likelihood, Day_{maximum probability}, is determined according to:

$$\text{Max} \left(\text{prob} \left({\text{day}}_{0}\right) , \text{prob} \left({\text{day}}_{1}\right) , \text{prob} \left({\text{day}}_{2}\right) , \text{prob} \left({\text{day}}_{3}\right) , \text{prob} \left({\text{day}}_{4}\right)\right)$$

According to one embodiment, the step of determining the threshold in the model parameter of the generated statistical model can be performed by, firstly, clustering the community of voluntary participants according to a model parameter of the generated statistical model. And, secondly, determining a plurality of clusters, each cluster including one or more participants self-reporting a similar health condition or a wellness status indication.

According to one embodiment, in the personal fertility analytics application, the personal health event that can be predicted according to process 400 can be one of an onset of menstruation, an ovulation date, a fertility window, pain before menstruation, or a combination thereof.

According to one embodiment, in the personal health pregnancy analytics application, the personal health event that can be predicted according to process 400 can be one of a pregnancy due date, a fetal developmental milestone, fetal distress, pregnancy complications, post-partum outcome, maternal health, newborn health characteristics, gender, or a combination thereof. As such, as discussed herein, an advertisement related to products associated with the personal health event can be provided to the user via the systems and methods discussed herein. For example, upon a detection/dctermination of a pregnancy due date, advertisements may be served to the user (via the implemented device) related to pregnancy medications, and/or other products/services that are related to such event.

### Personalized Interpretation of Clinical Guidelines

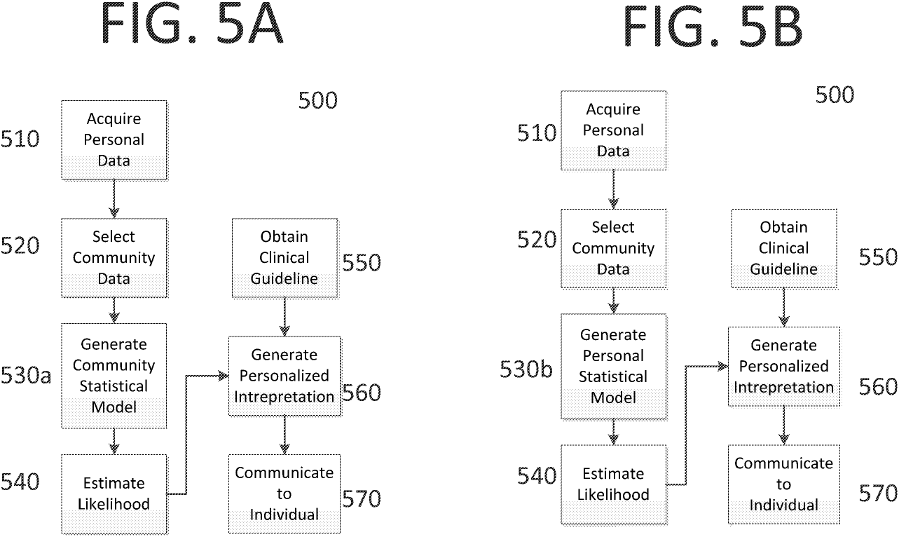

Now turning to FIGS. 5A and 5B, process 500 is provided for generating a personalized interpretation of clinical guidelines. According to one embodiment, steps 510, 520, 530, and 540 can be performed as describe above with respect to steps 410, 420, 430, and 440 of process 400. As such, the personalized interpretation of clinical guidelines takes, as an input, the estimated likelihood of a particular health event from step 540. Another information input can be from clinical guidelines for the health event. As shown, in step 550, a clinical guideline associated with the health event can be obtained through a number of conventional means. For example, journal-published clinical guidelines are typically generated from expert analyzed clinical trials and the associated dataset collected from a clinical setting.

Next, in step 560, a personalized interpretation of the clinical guidelines can be generated based from the estimated likelihood of the health event. In particular, a health attribute related to the health event can be predicted as function of the self-reported health attributes.

According to one embodiment, as shown in FIG. 5A, in step 530a, a statistical model describing a community can be generated in order to permit an estimate of the likelihood of the health event in step 540. According to another embodiment, as shown in FIG. 5B, in step 530b, a statistical model describing an individual participant can be generated for the subsequent step 540.

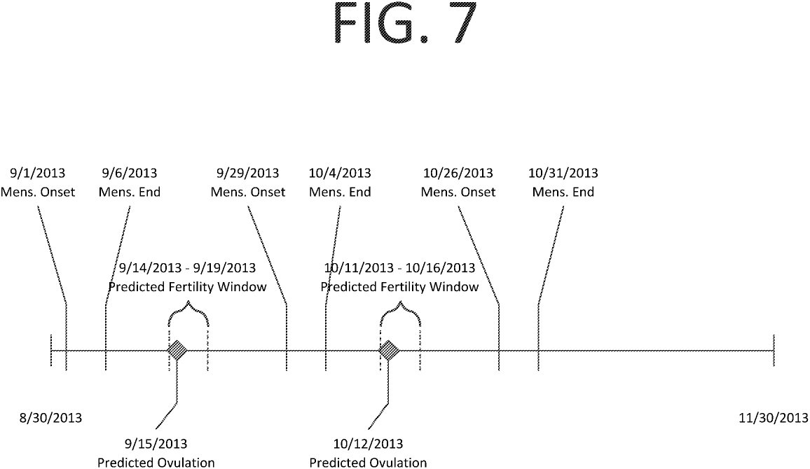

According to one embodiment, in a fertility predictive application shown in FIG. 7, a prediction of ovulation date can be further personalized using the generated statistical model. For example, a clinical guideline for predicting a window of fertility may be based on a 28 day menstruation cycles and a five (5) day menstruation duration, and a fertility window can be predicted by projecting forward 28 days from the onset of menstruation, to the onset of the next menstruation date. From the next menstruation date, a fertility window can be predicted by projecting backwards, 14 days, from the next onset of menstruation. For illustrative purposes, a person with regular menstruation can be provided with a personalized prediction. In particular, a self-reported onset of menstruation can be used project forward to a next onset of menstruation, and self-reported subsequent onset of menstruation can be used to confirm that the menstruation is regular. According to another embodiment, the fertility window prediction can be further personalized according to self-reported health attributes, i.e. a self-reported cervical fluid. For example, a self-reported cervical fluid indicating onset of a fertility window can be used to adjust a personalized model of when the onset of the fertility window can be back projected from the next onset of menstruation.

According to another embodiment, a pregnancy predictive application can be provided based on personalized statistical model of self-reported health attributes. For example, a due date can be predicted from a set of self-reported pregnancy symptoms.

Finally, in step 570, the personalized information concerning the health event can be communicated to the individual participant. In particular, the information concerning the health event can be the generated personal interpretation of the clinical guideline.

According to one embodiment, in step 570, the personalized information concerning the health event can be communicated to the participant when the estimate likelihood exceeds a predetermined threshold value. In particular, step 570 can further include a step of determining a threshold in the estimated likelihood of the personal health attribute. The threshold can represent a likelihood of the personal health attribute above which a predetermined health action can be beneficial to the individual participant. Step 570 can also include a step of recommending, to the individual participant, the predetermined health action when the estimated likelihood exceeds the determined threshold. As discussed above, an advertisement may also be served to the user based at least in part upon the clinical guideline, health event, predetermined health action and/or personal health attribute, and the like, and/or any combination thereof.

### Knowledge Representation

According to one embodiment, the obtained clinical guidelines can be represented in a database, and the personalized recommendation can be further represented as a look-up table. As shown below in Table 1, a plurality of self-report health attributes can be used to predict a future health event.

TABLE 1

| Conditions | 1 | 2 |
|---|---|---|
| 0 | 10 | 2 |
| THIS ROW IS CONTENT DISPLAYED WHEN CONDITIONS ACTIVATED | You reported high blood pressure and other symptoms that may indicate preeclampsia. You should call your doctor. | You reported high blood pressure and vision changes, which may indicate preeclampsia. You should call your doctor. |
| NOTE: All of these notes should instruct a client experiencing these symptoms to consult with their doctor. These are just possible warning signs to help catch a complication early by their own submission of symptoms. | Pre-eclampsia, eclampsia, and HELLP Syndrome T2: http://www.preeclampsia.org | Pre-eclampsia, eclampsia, and HELLP Syndrome T2: http://www.preeclampsia.org |
| weight gain: low | 0 | 0 |
| weight gain: rapid | 1 | 0 |
| weight loss | 0 | 0 |
| weight gain: low | 0 | 0 |
| weight gain: rapid | 1 | 0 |
| weight loss | 0 | 0 |
| blood pressure: high | 1 | 1 |
| blood pressure: low | 0 | 0 |
| activity: low | 0 | 0 |
| activity: high | 0 | 0 |
| T1 Anxious | 0 | 0 |
| T1 Depressed | 0 | 0 |
| T1 Stressed | 0 | 0 |
| T1 abdominal aching and pains | 0 | 0 |
| T1 Nausea | 0 | 0 |
| T1 Swelling | 0 | 0 |
| T1 Backache | 0 | 0 |
| T1 Pelvic Discomfort and Pressure | 0 | 0 |
| T1 Vaginal Spotting/Bleeding | 0 | 0 |
| T1 Cold and Flu Symptoms | 0 | 0 |
| T1 Fainting | 0 | 0 |
| T1 Fatigue/Exhaustion | 0 | 0 |
| T1 Mood Swings | 0 | 0 |
| T2 Anxious | 0 | 0 |
| T2 Depressed | 0 | 0 |
| T2 Stressed | 0 | 0 |
| T2 abdominal aching and pains | 1 | 0 |
| T2 Appetite Increase | 0 | 0 |
| T2 Contractions (Braxton-Hicks) | 0 | 0 |
| T2 Nausea | 1 | 0 |
| T2 Backache | 0 | 0 |
| T2 Swelling | 1 | 0 |
| T2 Shortness of Breath | 0 | 0 |
| T2 Dizzy | 1 | 0 |
| T2 Headache | 1 | 0 |
| T2 Nose Congestion | 0 | 0 |
| T2 Vision Changes | 1 | 1 |
| T2 Bloody show, Passing of Mucous Plug | 0 | 0 |
| T2 Cervical Dilation and Effacement | 0 | 0 |
| T2 Frequent Urination | 0 | 0 |
| T2 Hemorrhoids | 0 | 0 |
| T2 Pelvic Discomfort and Pressure | 0 | 0 |
| T2 Ruptured Membranes | 0 | 0 |
| T2 Urinary Incontinence | 0 | 0 |
| T2 Vaginal Discharge | 0 | 0 |
| T2 Vaginal Spotting/Bleeding | 0 | 0 |
| T2 Cold and Flu Symptoms | 0 | 0 |
| T2 Fainting | 0 | 0 |
| T2 Fatigue/Exhaustion | 0 | 0 |
| T2 Increase Energy | 0 | 0 |
| T2 PMS Symptoms | 0 | 0 |
| T3 Anxious | 0 | 0 |
| T3 Depressed | 0 | 0 |
| T3 Stressed | 0 | 0 |
| T3 abdominal aching and pains | 0 | 0 |
| T3 Appetite Increase | 0 | 0 |
| T3 Contractions (Braxton-Hicks) | 0 | 0 |
| T3 Nausea | 0 | 0 |
| T3 Swelling | 0 | 0 |
| T3 Shortness of Breath | 0 | 0 |
| T3 Dizzy | 0 | 0 |
| T3 Headache | 0 | 0 |
| T3 Nose Congestion | 0 | 0 |
| T3 Vision Changes | 0 | 0 |
| T3 Bloody show, Passing of Mucous Plug | 0 | 0 |
| T3 Cervical Dilation and Effacement | 0 | 0 |
| T3 Frequent Urination | 0 | 0 |
| T3 Pelvic Discomfort and Pressure | 0 | 0 |
| T3 Ruptured Membranes | 0 | 0 |
| T3 Urinary Incontinence | 0 | 0 |
| T3 Vaginal Discharge | 0 | 0 |
| T3 Vaginal Spotting/Bleed ing | 0 | 0 |
| T3 Cold and Flu Symptoms | 0 | 0 |
| T3 Fainting | 0 | 0 |
| T3 Fatigue/Exhaustion | 0 | 0 |

As shown, if a participant has reported high blood pressure and other symptoms, i.e. self-reported health attributes that may indicate preeclampsia, a doctor visit can be recommended to the participant. According to one embodiment, a plurality of self-reported attributes can be aggregated to provide a summary recommendation. For example, as shown in Table 1, each of the self-reported health attributes can be represented by a Boolean variable (see columns 2 and 3).

### Personalized Information Intermediation

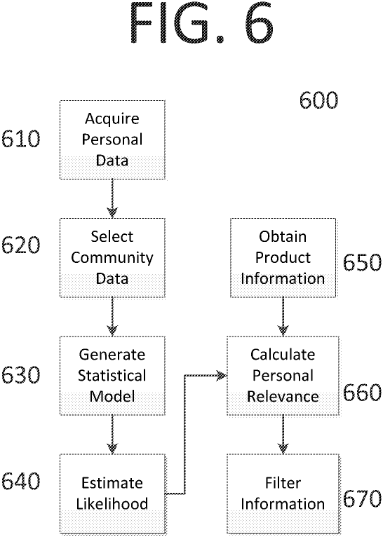

Turning to FIG. 6, process 600 is provided for intermediating personalized health information to participants of self-reported health information.

According to one embodiment, as shown in FIG. 6, steps 610, 620, 630, and 640 can be performed as describe above with respect to steps 410, 420, 430, and 440 of process 400. As such, the intermediation of personalized health information takes, as an input, the estimated likelihood of a particular health event from step 640. The process 600 can include a step 660 for determining a personal relevance for a general collection of wellness information, which can be obtained in step 650.

According to one embodiment, the step 660 further includes a step 670 for selecting a personalized collection from the general collection, from step 650, based on the estimated likelihood, and the thus personalized collection can be provided to the individual participant.

According to one embodiment, a wide range of personal health information can be intermediated to the individual participants according to the process 600. For example, the process 600 can be performed to provide a personalized collection of commercial product information, product offering from a third party merchants, or collections of personal wellness recommendations. For example, such information may be provided to a third party service/product provider, where products/services may be provided to the user based on such information (e.g., ads and/or promotions - or information related to services products correlated with such personalized collection of information), as discussed above.

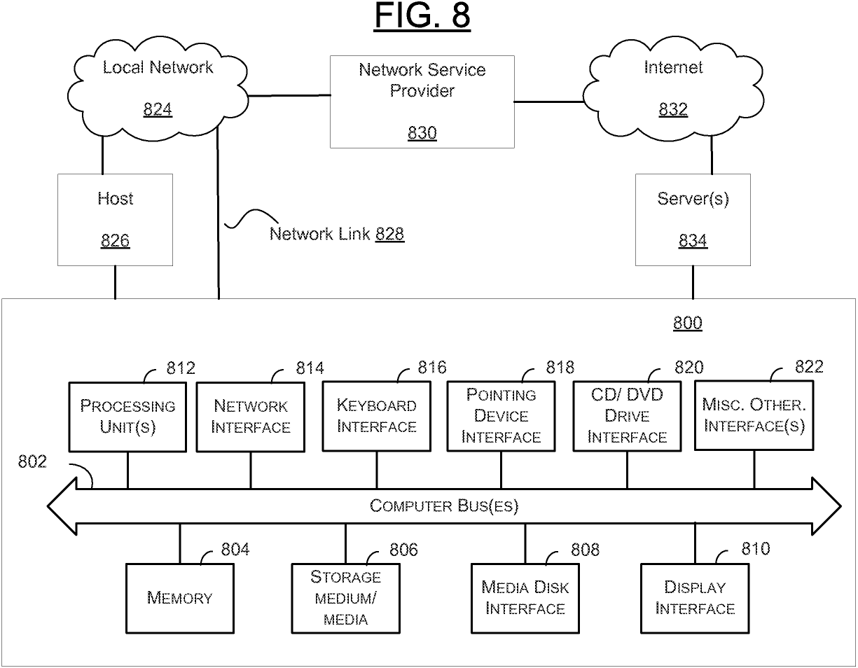

As shown in FIG. 8, internal architecture 800 includes one or more processing units, processors, or processing cores, (also referred to herein as CPUs) 812, which interface with at least one computer bus 802. Also interfacing with computer bus 802 are computer-readable medium, or media, 806, network interface 814, memory 804, e.g., random access memory (RAM), run-time transient memory, read only memory (ROM), media disk drive interface 820 as an interface for a drive that can read and/or write to media including removable media such as floppy, CD-ROM, DVD, media, display interface 810 as interface for a monitor or other display device, keyboard interface 816 as interface for a keyboard, pointing device interface 818 as an interface for a mouse or other pointing device, and miscellaneous other interfaces not shown individually, such as parallel and serial port interfaces and a universal serial bus (USB) interface.

Memory 804 interfaces with computer bus 802 so as to provide information stored in memory 804 to CPU 812 during execution of software programs such as an operating system, application programs, device drivers, and software modules that comprise program code, and/or computer executable process steps, incorporating functionality described herein, e.g., one or more of process flows described herein. CPU 812 first loads computer executable process steps from storage, e.g., memory 804, computer readable storage medium/media 806, removable media drive, and/or other storage device. CPU 812 can then execute the stored process steps in order to execute the loaded computer-executable process steps. Stored data, e.g., data stored by a storage device, can be accessed by CPU 812 during the execution of computer-executable process steps.

Persistent storage, e.g., medium/media 806, can be used to store an operating system and one or more application programs. Persistent storage can also be used to store device drivers, such as one or more of a digital camera driver, monitor driver, printer driver, scanner driver, or other device drivers, web pages, content files, playlists and other files. Persistent storage can further include program modules and data files used to implement one or more embodiments of the present disclosure, e.g., listing selection module(s), targeting information collection module(s), and listing notification module(s), the functionality and use of which in the implementation of the present disclosure are discussed in detail herein.

Network link 828 typically provides information communication using transmission media through one or more networks to other devices that use or process the information. For example, network link 828 may provide a connection through local network 824 to a host computer 826 or to equipment operated by a Network or Internet Service Provider (ISP) 830. ISP equipment in turn provides data communication services through the public, worldwide packet-switching communication network of networks now commonly referred to as the Internet 832.

A computer called a server host 834 connected to the Internet 832 hosts a process that provides a service in response to information received over the Internet 832. For example, server host 834 hosts a process that provides information representing video data for presentation at display 810. It is contemplated that the components of system 800 can be deployed in various configurations within other computer systems, e.g., host and server.

At least some embodiments of the present disclosure are related to the use of computer system 800 for implementing some or all of the techniques described herein. According to one embodiment, those techniques are performed by computer system 800 in response to processing unit 812 executing one or more sequences of one or more processor instructions contained in memory 804. Such instructions, also called computer instructions, software and program code, may be read into memory 804 from another computer-readable medium 806 such as storage device or network link. Execution of the sequences of instructions contained in memory 804 causes processing unit 812 to perform one or more of the method steps described herein. In alternative embodiments, hardware, such as ASIC, may be used in place of or in combination with software. Thus, embodiments of the present disclosure are not limited to any specific combination of hardware and software, unless otherwise explicitly stated herein.

The signals transmitted over network link and other networks through communications interface, carry information to and from computer system 800. Computer system 800 can send and receive information, including program code, through the networks, among others, through network link and communications interface. In an example using the Internet, a server host transmits program code for a particular application, requested by a message sent from computer, through Internet, ISP equipment, local network and communications interface. The received code may be executed by processor 802 as it is received, or may be stored in memory 804 or in storage device or other non-volatile storage for later execution, or both.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

For the purposes of this disclosure the term “user”, “subscriber” “consumer” or “customer” should be understood to refer to a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods arc not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

1-16. (canceled)

17. A system comprising:
a non-transitory computer-readable medium storing computer-executable program instructions; and
a processing device communicatively coupled to the non-transitory computer-readable medium for executing the computer-executable program instructions, wherein executing the computer-executable program instructions configures the processing device to perform operations comprising:
obtaining, from each of a plurality of devices, a respective dataset comprising a set of health attributes comprising a menstrual cycle type and menstrual period dates associated with a user of the respective device;
generating, from each of the datasets, an additional subset of the datasets by:
grouping one or more of the datasets based on an associated menstruation regularity and age, thereby forming a subset of the datasets; and
further grouping, within the subset of the datasets, one or more of the plurality of datasets based on associated menstrual cycle length and menstrual duration, thereby forming the additional subset of the datasets;
generating, from the additional subset of the datasets, a statistical model that predicts one or more occurrences of a health event within datasets;
receiving, from a first device, a time sequence of health attributes comprising a menstrual cycle type and menstrual period dates;
generating, from the time sequence of health attributes and the statistical model, a first statistical model;
estimating, from the first statistical model and for each day in a predetermined set of consecutive days, a probability of the health event occurring, and wherein estimating the probability comprises:
determining a threshold in a model parameter of the first statistical model, the threshold separating a sequence of health attributes likely to be associated with a personal health attribute from a sequence of health attributes unlikely to be associated with the personal health attribute;
estimating the model parameter of the first statistical model according to the time sequence of health attributes; and
classifying the time sequence of health attributes according to the determined threshold;
generating, in accordance with a clinical guideline that is associated with the health event and predicts a regular occurrence of the health event, a day of the predetermined set of consecutive days having a maximum likelihood among the predetermined set of consecutive days; and
providing, to the first device, an indication that the health event will likely occur on the day.

18. The system of claim 17, wherein the health event is one or more of: an onset of menstruation, an ovulation date, a fertility window, pain before menstruation.

19. The system of claim 17, further comprising:
predicting a health attribute related to the health event as function of the health attributes; and
transmitting, to the first device, a recommendation comprising a predetermined health action based on the predicted health attribute.

20. The system of claim 17, wherein obtaining the respective datasets further comprises:
displaying, periodically, on one or more devices of the plurality of devices, a first set of questions designed to obtain a time series of variable health attributes;
displaying, periodically, on the one or more devices, a second set of questions, the second set of questions being designed to obtain a status of a past or current health event;
receiving, from the one or more devices, a plurality of answers to the first and second set of questions; and
providing, to the one or more devices, a personal feedback based on one of the plurality of answers.

21. The system of claim 17, wherein determining the threshold in the model parameter of the first statistical model comprises:
clustering the devices of the plurality of devices according to the model parameter of the generated statistical model; and
determining a plurality of clusters, each cluster including one or more devices reporting a similar health condition or a wellness status indication.

22. The system of claim 17, wherein the health attribute is selected is selected from the group consisting of a mood quality, a menstrual cycle type, a menstrual period length, date of last period, menstrual period dates, spotting dates, cervical fluid quality, intercourse dates, ovulation test results, pregnancy test results, body basal temperature, number of steps walked, health quality, weight, medications taken, nutrition consumed, time slept, blood pressure, activity, other relevant notes, and a combination thereof.

23. The system of claim 17, wherein the health attribute is selected from the group consisting of a pregnancy due date, a fetal developmental milestone, fetal distress, pregnancy complications, post-partum outcome, maternal health, newborn health characteristics, gender, and a combination thereof.

24. A method of predicting a health event, the method comprising:
obtaining, from each of a plurality of devices, a respective dataset comprising a set of health attributes comprising a menstrual cycle type and menstrual period dates associated with a user of the respective device;
generating, from each of the datasets, an additional subset of the datasets by:
grouping one or more of the datasets based on an associated menstruation regularity and age, thereby forming a subset of the datasets; and
further grouping, within the subset of the datasets, one or more of the plurality of datasets based on associated menstrual cycle length and menstrual duration, thereby forming the additional subset of the datasets;
generating, from the additional subset of the datasets, a statistical model that predicts one or more occurrences of the health event within datasets;
receiving, from a first device, a time sequence of health attributes comprising a menstrual cycle type and menstrual period dates;
generating, from the time sequence of health attributes and the statistical model, a first statistical model;
estimating, from the first statistical model and for each day in a predetermined set of consecutive days, a probability of the health event occurring, and wherein estimating the probability comprises:
determining a threshold in a model parameter of the first statistical model, the threshold separating a sequence of health attributes likely to be associated with a personal health attribute from a sequence of health attributes unlikely to be associated with the personal health attribute;
estimating the model parameter of the first statistical model according to the time sequence of health attributes; and
classifying the time sequence of health attributes according to the determined threshold;
generating, in accordance with a clinical guideline that is associated with the health event and predicts a regular occurrence of the health event, a day of the predetermined set of consecutive days having a maximum likelihood among the predetermined set of consecutive days; and
providing, to the first device, an indication that the health event will likely occur on the day.

25. The method of claim 24, wherein the health event is one or more of: an onset of menstruation, an ovulation date, a fertility window, pain before menstruation.

26. The method of claim 24, further comprising:
predicting a health attribute related to the health event as function of the health attributes; and
transmitting, to the first device, a recommendation comprising a predetermined health action based on the predicted health attribute.

27. The method of claim 24, wherein obtaining the respective datasets further comprises:
displaying, periodically, on one or more devices of the plurality of devices, a first set of questions designed to obtain a time series of variable health attributes;
displaying, periodically, on the one or more devices, a second set of questions, the second set of questions being designed to obtain a status of a past or current health event;
receiving, from the one or more devices, a plurality of answers to the first and second set of questions; and
providing, to the one or more devices, a personal feedback based on one of the plurality of answers.

28. The method of claim 24, wherein determining the threshold in the model parameter of the first statistical model comprises:
clustering the devices of the plurality of devices according to the model parameter of the generated statistical model; and
determining a plurality of clusters, each cluster including one or more devices reporting a similar health condition or a wellness status indication.

29. The method of claim 24, wherein the health attribute is selected is selected from the group consisting of a mood quality, a menstrual cycle type, a menstrual period length, date of last period, menstrual period dates, spotting dates, cervical fluid quality, intercourse dates, ovulation test results, pregnancy test results, body basal temperature, number of steps walked, health quality, weight, medications taken, nutrition consumed, time slept, blood pressure, activity, other relevant notes, and a combination thereof.

30. A non-transitory computer-readable storage medium storing computer-executable program instructions, wherein when executed by a processing device, the computer-executable program instructions cause the processing device to perform operations comprising:
obtaining, from each of a plurality of devices, a respective dataset comprising a set of health attributes comprising a menstrual cycle type and menstrual period dates associated with a user of the respective device;
generating, from each of the datasets, an additional subset of the datasets by:
grouping one or more of the datasets based on an associated menstruation regularity and age, thereby forming a subset of the datasets; and
further grouping, within the subset of the datasets, one or more of the plurality of datasets based on associated menstrual cycle length and menstrual duration, thereby forming the additional subset of the datasets;
generating, from the additional subset of the datasets, a statistical model that predicts one or more occurrences of a health event within datasets;
receiving, from a first device, a time sequence of health attributes comprising a menstrual cycle type and menstrual period dates;
generating, from the time sequence of health attributes and the statistical model, a first statistical model;
estimating, from the first statistical model and for each day in a predetermined set of consecutive days, a probability of the health event occurring, and wherein estimating the probability comprises:
determining a threshold in a model parameter of the first statistical model, the threshold separating a sequence of health attributes likely to be associated with a personal health attribute from a sequence of health attributes unlikely to be associated with the personal health attribute;
estimating the model parameter of the first statistical model according to the time sequence of health attributes; and
classifying the time sequence of health attributes according to the determined threshold;
generating, in accordance with a clinical guideline that is associated with the health event and predicts a regular occurrence of the health event, a day of the predetermined set of consecutive days having a maximum likelihood among the predetermined set of consecutive days; and
providing, to the first device, an indication that the health event will likely occur on the day.

31. The non-transitory computer-readable storage medium of claim 30, wherein the health event is one or more of: an onset of menstruation, an ovulation date, a fertility window, pain before menstruation.

32. The non-transitory computer-readable storage medium of claim 30, wherein the operations further comprise:
predicting a health attribute related to the health event as function of the health attributes; and
transmitting, to the first device, a recommendation comprising a predetermined health action based on the predicted health attribute.

33. The non-transitory computer-readable storage medium of claim 30, wherein obtaining the respective datasets further comprises:
displaying, periodically, on one or more devices of the plurality of devices, a first set of questions designed to obtain a time series of variable health attributes;
displaying, periodically, on the one or more devices, a second set of questions, the second set of questions being designed to obtain a status of a past or current health event;
receiving, from the one or more devices, a plurality of answers to the first and second set of questions; and
providing, to the one or more devices, a personal feedback based on one of the plurality of answers.

34. The non-transitory computer-readable storage medium of claim 30, wherein determining the threshold in the model parameter of the first statistical model comprises:
clustering the devices of the plurality of devices according to the model parameter of the generated statistical model; and
determining a plurality of clusters, each cluster including one or more devices reporting a similar health condition or a wellness status indication.
